(19) United States
(12) Patent Application Publication
(10) Pub. No.: US 2026/0114902 A1
(43) Pub. Date: Apr. 30, 2026

(54) Access Device for Surgical Procedures

(71) Applicant: Fetal Therapy Technologies, LLC, Baltimore, MD (US)

(72) Inventors: Selena Shirkin, Baltimore, MD (US); Eric McAlexander, Severna Park, MD (US); Gloria Kalnitskaya, Gaithersburg, MD (US); Ayeeshi Poosarla, Fremont, CA (US); Mariah Snelson, Baltimore, MD (US); Alice Yu, San Diego, CA (US); Ahmet Baschat, Towson, MD (US)

(73) Assignee: Fetal Therapy Technologies, LLC, Baltimore, MD (US)

(21) Appl. No.: 19/478,801

(22) PCT Filed: Mar. 20, 2025
(86) PCT No.: PCT/US2025/020771

§ 371 Date: Oct. 24, 2025

Related U.S. Application Data
(60) Provisional application No. 63/567,984, filed on Mar. 21, 2024.

(51) Int. Cl. A61B 17/42; A61B 17/00; A61B 17/34

(52) U.S. Cl. CPC A61B 17/42; A61B 17/34; A61B 17/3403; A61B 17/3496; A61B 17/3498; A61B 2017/00862; A61B 2017/3413; A61B 2017/3486; A61B 2017/4216

(57) ABSTRACT

The present disclosure provides an access device for surgical procedures. The access device includes an obturator comprising a cylindrical rod with a first end having a tip and a second end, a disc-shaped stopper attachable to the second end at a first position, and an end cap attachable to the second end at a second position further from the tip than the first position. The device also includes a sheath having an elastic body with a radially expanding tube and a set of echogenic reinforcing ribs extending axially along and protruding radially inward from the inner surface of the tube. The ribs act as guide rails for the obturator, and the tube has a smooth outer surface for sliding through tissue. The sheath is configured to pass the obturator through the tube along the ribs until the stopper rests against the tube.

### CROSS-REFERENCE TO RELATED APPLICATION(S)

This application is a PCT international application that claims the benefit of U.S. Provisional Application No. 63/567,984, filed Mar. 21, 2024, entitled “PLATFORM ACCESS DEVICE FOR MICROSURGICAL PROCEDURES COMPRISING A RADIALLY EXPANDING PORT SHEATH AND DEPTH-CONTROL OBTURATOR.”

The contents of each of the above referenced applications are hereby incorporated by reference in their entirety.

### TECHNICAL FIELD

Various aspects of the present disclosure relate generally to surgical procedures, more particularly, to systems and methods for an access device for (micro) surgical procedures that comprises a radially expanding sheath and a depth-control obturator.

### BACKGROUND

Minimally invasive surgical procedures, such as laparoscopic and robotic-assisted surgeries, rely heavily on the use of access devices that provide a pathway for surgical instruments to reach a targeted tissue, body organ, etc. One common type of access device comprises a sheath and an obturator. The sheath establishes and maintains access to a surgical site, while the obturator, typically a solid or hollow rod, is inserted into the sheath to facilitate the sheath/obturator introduction through tissue layers, such as the abdominal wall. Once the sheath is in place, the obturator is removed, leaving the sheath as a conduit for surgical instruments.

However, there are a number of challenges and potential problems associated with the use of such access devices. For example, the insertion of the obturator and the sheath can cause unintended damage to surrounding tissues or underlying organs; this problem is relevant when the surgical site is difficult to visualize or where a surgeon must exert significant force to advance the access device through the tissue(s). Furthermore, once the obturator is removed, the sheath may not have sufficient fixation and strength in the tissues, which can cause it to inadvertently displace or kink during patient movement (when tissue layers may shift and create lateral shear forces); this instability can interfere with the surgical procedure and increase the risk of complications. Some other potential problems peculiar to the current designs of the access devices also include insufficient ease of use and versatility.

Another challenge with the access devices, especially at small scales, is that there are currently no options to “size up” an access port in the middle of surgery-if a surgeon wanted to size up their access, they would need to first remove the existing sheath. This entails placing a guidewire through the existing sheath, removing that sheath, and placing a larger sheath over top of that guidewire. This process takes time and uses extra instruments in the operating room. The act of sizing up the sheath access, especially in delicate anatomies like the uterine environment, can cause unintended damage and trauma to the surrounding tissues.

A further challenge with the current port systems is what's known as a “port step”. This is when a port sheath is physically cut down or shortened to fit the anatomy that it will be used in. Because the sheath is cut down, it loses its tapered edge and thus no longer fits flush against the obturator. Because these instruments do not fit flush with one another after being modified, there is a gap between the sheath and obturator. Cutting the sheath to a shorter length may also leave a jagged edge instead of the original tapered one. These characteristics create a “port step” which causes tissue to snag onto the access system during insertion. If this access system with a port step were inserted into the delicate uterine environment, it would snag tissue from the uterus, causing damage during insertion.

In procedures that take place in the uterine environment, the current access sheath that is used is not strong enough to withstand the contractile forces of the uterus. During a fetal surgical procedure, the empty sheath can bend or ‘kink’ when uterine contractions take place. This ‘kinking’ of the sheath can cause plastic deformation to the walls of the sheath in the form of a crease. This crease could increase the likelihood that an inserted instrument would unintentionally pierce the walls of the sheath during insertion.

The present disclosure is directed to overcoming one or more of these above-referenced challenges.

### SUMMARY OF THE DISCLOSURE

According to certain aspects of the disclosure, systems, methods, and computer readable memory are disclosed for an access device.

In some cases, an access device for surgical procedures may include: an obturator, wherein the obturator is a cylindrical rod that has a first end and a second end, and the first end has a tip; a disc-shaped stopper, wherein the disc-shaped stopper is configured to be attached to the second end of the obturator at a first position along an axial length of the second end; an end cap, wherein the end cap is configured to be attached to the second end of the obturator at a second position along the axial length of the second end, and the second position is further away from the tip of the first end than the first position; and a sheath having an elastic body, wherein the elastic body includes: a radially expanding tube, and a set of echogenic reinforcing ribs extending axially along an inner surface of the radially expanding tube and protruding radially inwardly from the inner surface of the radially expanding tube, wherein the set of reinforcing ribs are configured to act as guide rails for the obturator, maintain the structural integrity of the port during shear/kinking forces, and be visible on ultrasound, and the radially expanding tube has a smooth outer surface to cause the radially expanding tube to slide through a tissue of interest, and wherein the sheath is configured to pass the obturator through the radially expanding tube along the set of reinforcing ribs until the disc-shaped stopper attached to the second end of the obturator rests against the radially expanding tube.

In some cases, an obturator may include: a cylindrical rod that has a first end and a second end, wherein the first end has a tip; a disc-shaped stopper, wherein the disc-shaped stopper is configured to be attached to the second end of the obturator at a first position along an axial length of the second end; and an end cap, wherein the end cap is configured to be attached to the second end of the obturator at a second position along the axial length of the second end, and the second position is further away from the tip of the first end than the first position.

In some cases, an access device for surgical procedures may include: a sheath having an elastic body, wherein the elastic body includes: a radially expanding tube, and a set of echogenic reinforcing ribs extending axially along an inner surface of the radially expanding tube and protruding radially inwardly from the inner surface of the radially expanding tube, wherein the set of reinforcing ribs are configured to act as guide rails for an obturator and/or surgical devices and are visible on ultrasound imaging, and the radially expanding tube has a smooth outer surface to cause the radially expanding tube to slide through a tissue of interest, and wherein the sheath is configured to pass the obturator and/or the surgical devices through the radially expanding tube along the set of reinforcing ribs.

In some cases, a method for manufacturing a sheath of an access device may include: preparing a mold for a sheath, wherein the mold has an inner longitudinal channel shaped as a radially expanding tube of the sheath, the inner longitudinal channel has a longitudinal axis, and the mold is configured to be disassembled into two halves such that the inner longitudinal channel is divided in half along the longitudinal axis of the channel; preparing a mold plunger, wherein the mold plunger is shaped to be inserted into the inner longitudinal channel of the mold, and the mold plunger includes longitudinal grooves each configured to correspond to a shape of one of the set of reinforcing ribs; inserting the mold plunger into the mold; injecting a self-curing polymer into a space between the mold and the mold plunger; and after the self-curing polymer is cured, removing the mold plunger and disassembling the mold into the two halves, thereby releasing the sheath.

In some cases, a method for performing a surgical procedure by using an access device may include: inserting an obturator into a sheath such that a disc-shaped stopper attached to a second end of the obturator rests against an elastic body of the sheath; providing access to a surgical site of interest by making a cut on a human body using the obturator inserted into the sheath; removing the obturator from the sheath, while holding the sheath relative to the surgical site; and inserting a medical instrument of interest into the surgical site through the sheath.

Additional objects and advantages of the disclosed technology will be set forth in part in the description that follows, and in part will be apparent from the description, or may be learned by practice of the disclosed technology.

It is to be understood that both the foregoing general description and the following detailed description are exemplary and explanatory only and are not restrictive of the disclosed technology, as claimed.

### BRIEF DESCRIPTION OF THE DRAWINGS

The accompanying drawings, which are incorporated in and constitute a part of this specification, illustrate various exemplary aspects and together with the description, serve to explain the principles of the disclosed technology.

FIG. 1 illustrates an isometric view of an access device, according to aspects of the present disclosure.

FIG. 2A illustrates an orthogonal side view of a portion of an access device, according to an embodiment.

FIG. 2B illustrates a section view of a port sheath, according to aspects of the present disclosure.

FIG. 2C shows an exploded view of a port body, according to an embodiment.

FIG. 3A illustrates a side view of a port sheath with dimensional characteristics, according to aspects of the present disclosure.

FIG. 3B illustrates a section view of a port sheath assembly, according to an embodiment.

FIG. 3C illustrates a side view of a portion of the access device, according to aspects of the present disclosure.

FIG. 4A illustrates an orthogonal view of a first rib type, according to an embodiment.

FIG. 4B illustrates a detail view of a portion of the access device, according to aspects of the present disclosure.

FIG. 4C illustrates a section view of a second type of rib configuration, according to an embodiment.

FIG. 4D illustrates an axial view of a second type of rib, according to aspects of the present disclosure.

FIG. 5 illustrates an orthogonal view of an obturator, according to an embodiment.

FIG. 6A illustrates an orthogonal view of an offset bevel tip, according to aspects of the present disclosure.

FIG. 6B illustrates a detailed view of the faces and edges of an offset bevel tip, according to an embodiment.

FIG. 7 illustrates different views of a disc-shaped stopper assembly, according to aspects of the present disclosure.

FIG. 8 illustrates orthogonal views of a cap, according to an embodiment.

FIGS. 9A-9C illustrate views of a first half of a mold assembly, according to aspects of the present disclosure.

FIGS. 9D-9F illustrate views of a second half of a mold assembly, according to an embodiment.

FIGS. 10A-10C illustrate views of a plunger assembly used in manufacturing, according to aspects of the present disclosure.

FIG. 11 illustrates a section view of an access device with fluid exchange capabilities, according to an embodiment.

FIG. 12 illustrates expansion states of a radially expanding port system, according to aspects of the present disclosure.

FIG. 13 illustrates a side view of an access device with a balloon anchoring mechanism, according to an embodiment.

### DETAILED DESCRIPTION

Various aspects of the present disclosure relate generally to an access device.

The present disclosure describes an access device for (micro) surgical procedures that addresses the above-mentioned challenges and provides improved safety, stability, ease of use, and versatility.

According to a first aspect, an access device for surgical procedures is provided, which comprises an obturator, a disc-shaped stopper, an end cap, and a sheath. The obturator is made as a cylindrical rod that has a first end and a second end, with the first end being provided with a tip. The disc-shaped stopper and the end cap are configured to be successively attached to the second end of the obturator. The sheath has an elastic body configured to pass the obturator therethrough until the disc-shaped stopper attached to the second end of the obturator rests against the elastic body. The elastic body of the sheath is made as a radially expanding tube provided with reinforcing ribs extending longitudinally along an inner surface of the radially expanding tube. The reinforcing ribs are configured to act as guide rails for the obturator. The radially expanding tube has an outer surface that is made smooth to cause the radially expanding tube to slide through a tissue of interest with minimal friction. In the access device so configured, the depth of the obturator insertion can be effectively adjusted by appropriately attaching the stopper to the second end of the obturator. This in turn minimizes tissue trauma during the surgical procedure. Furthermore, the presence of the reinforcing ribs makes the sheath structure more robust and stable, preventing kinking of the sheath during surgery and stopping instruments from piercing through the side of the sheath. The presence of reinforcing ribs also make the port sheath visible on ultrasound imaging. It should also be noted that the access device according to the present disclosure allows surgeons to size up the sheath access as needed in the middle of surgery due to its radially expanding capabilities.

In one exemplary embodiment of the first aspect, the second end of the cylindrical rod has a threaded portion. In this embodiment, each of the disc-shaped stopper and the end cap are configured to be threaded onto the threaded portion. By using this threaded connection, the obturator penetration depth (relative to the sheath) can be controlled more easily and effectively.

In one exemplary embodiment of the first aspect, the threaded portion of the cylindrical rod has M2×0.4 threads, and the disc-shaped stopper has a central threaded hole passing therethrough and having M2×0.4 threads. This threaded configuration allows for finer tuning of the obturator penetration depth.

In one exemplary embodiment of the first aspect, the end cap is made as a compressed spherical knob having a dead threaded hole. The dead threaded hole has M2×0.4 threads. The end cap so configured can evenly distribute force across the surgeon's palm, allowing for a more precise application of force through the obturator.

In one alternative exemplary embodiment of the first aspect, the second end of the cylindrical rod is provided with an interlocking mechanism. In this embodiment, each of the disc-shaped stopper and the end cap are configured to be attached to the second end by the interlocking mechanism. The use of interlocking mechanism can provide more robust mechanical attachment of the stopper and the end cap to the obturator.

In one exemplary embodiment of the first aspect, the disc-shaped stopper has a circumferential chamfer on each edge and at least one concave engraved channel. This configuration of the stopper makes it easier to grip and work with gloves.

In one exemplary embodiment of the first aspect, the tip of the cylindrical rod is formed by four cutting edges comprising two long edges and two short edges, with the long edges alternating with the short edges so as to create a tip profile that provides a linear wound during insertion. In this tip profile, there are 4 cut faces—two “shallow” and two “deep” faces. Given this, the tip can act almost as a dagger, as the shallow faces can slice in a line, as opposed to piercing which occurs with identical faces.

In one exemplary embodiment of the first aspect, each of the reinforcing ribs has an elongated straight profile that is flared or chamfered at a point where the obturator enters the sheath and rounded at a point where the obturator exits the sheath. This rib profile allows inserted surgical instruments to slide along the ribs more efficiently.

In one exemplary embodiment of the first aspect, the radially expanding tube is made of self-curing polymer (e.g., elastic, flexible, low-friction, biocompatible polymer). Self-curing polymers exhibit proper elastic properties for surgical procedures.

In one exemplary embodiment of the first aspect, the access device further comprises a fluid exchange valve connected to the sheath at a point where the obturator enters the sheath. The fluid exchange valve is configured to fluid inlet and outlet through the sheath during the surgical procedure. Such a fluid (e.g., gas, saline, amniotic fluid) exchange valve may function while the tube is in its default (non-expanded) state or in its maximally expanded state.

In one exemplary embodiment of the first aspect, the radially expanding tube is configured to be self-anchored to a surrounding anatomy through a biocompatible inflatable balloon provided at a tip of the radially expanding tube. The balloon is configured to be inflated through the fluid exchange valve. Such a self-anchoring mechanism ensures more efficient fixation of the sheath in the surgical site of interest.

In one exemplary embodiment of the first aspect, the radially expanding tube has a beveled tip and a tapered wall. The beveled tip of the sheath fits flush against the obturator for a flat profile when inserted, creating a shallow slope during entry from the obturator tip to the sheath wall, while the tapered sheath wall allows for improved strength during expansion.

According to a second aspect, a method for manufacturing the sheath of the access device according to the first aspect is provided. The method starts with the step of preparing a mold for the sheath. The mold has an inner longitudinal channel shaped as the radially expanding sheath. The mold is configured to be disassembled into two halves such that the inner longitudinal channel is divided in half along the longitudinal axis of the channel. Next, the method goes on to the step of preparing a mold plunger shaped to be inserted into the inner longitudinal channel of the mold. The mold plunger comprises longitudinal grooves each configured to follow the shape of one of the reinforcing ribs. The reinforcing ribs are inserted into the grooves of the mold plunger before the mold plunger is inserted into the mold. Then, the method proceeds to the steps of inserting the mold plunger into the mold and injecting a self-curing polymer into the space between the mold and the mold plunger. After the self-curing polymer is cured, the next step is performed, in which the mold plunger is removed from the sheath and the mold is disassembled into the two halves, thereby releasing the sheath. By doing so, it is possible to manufacture the sheath structure that is more robust and stable compared to the prior art analogues, preventing kinking of the sheath during surgery and stopping instruments from piercing through the side of the sheath,

In one exemplary embodiment of the second aspect, one of the two halves of the mold comprises protruding hemispheres, and another of the two halves-concave hemispheres. The protruding hemispheres and the concave hemispheres are shaped to mate with each other when the two halves are assembled. By using such hemispheres, it is possible to provide more reliable attachment of the two halves of the mold during the manufacturing method.

According to a third aspect, a method for performing a surgical procedure by using the access device according to the first aspect is provided. The method starts with the step of inserting the obturator into the sheath such that the disc-shaped stopper attached to the second end of the obturator rests against the elastic body of the sheath. Then, the method proceeds to the step of providing access to a surgical site of interest by making a cut through the skin of a human body using a scalpel. The obturator and sheath are then inserted into the human body by pushing down on the end cap of the obturator until the sheath has entered the site of interest. Next, the method goes on to the step of removing the obturator from the sheath, thereby creating a pathway for a medical or surgical instrument of interest. After that, the method proceeds to the step of inserting the medical instrument of interest into the surgical site through the sheath. By using the access device in this way, it is possible to perform various surgical procedures more efficiently.

In one exemplary embodiment of the third aspect, the surgical procedure is a fetal surgical procedure, and the surgical site of interest comprises a uterus. In one alternative exemplary embodiment of the third aspect, the surgical site of interest comprises at least one of a vein and an arteria. Thus, the method according to the third aspect can be flexible in use and applied in different kinds of surgical procedures.

In some cases, the access device may significantly enhance microsurgical applications, particularly in the field of fetal surgery. While radial expansion sheaths have been utilized in laparoscopic procedures, traditional instruments designed for those applications are often too large and bulky for delicate fetal interventions. The access device may address this limitation by providing a more compact and adaptable solution tailored to the unique challenges of fetal surgery. Unlike laparoscopic technologies that create relatively large openings, which may be difficult to suture in the uterine environment and potentially lead to complications, the access device may leverage the natural elasticity of the uterus. By gently expanding the incision radially, the device may stretch the tissue rather than tear it, potentially eliminating the need for suturing. This approach may be particularly advantageous in the uterine environment, where conventional suturing techniques used in laparoscopic procedures may pose additional risks. The device's ability to adapt to the specific requirements of fetal surgery while minimizing tissue trauma may represent a significant advancement in microsurgical access techniques.

Thus, methods and systems of the present disclosure may be improvements to surgical procedures, and/or access devices for surgical procedures.

### 1. INTRODUCTION

In some cases, an access device for (micro) surgical procedures may include the following constructive elements, each with its own unique design features: an end cap, a stopper, an obturator, a sheath having ribs inside the sheath. The end cap is a rounded knob that easily conforms to the anatomy of the operator's palm. The end cap allows for an even distribution of force along the obturator as the operator pushes down on the obturator component. The stopper can be a stainless steel disc that screws up and down the threaded portion of the obturator by rotating along the vertical axis of the obturator. The obturator has a threaded portion along its length which both secures end cap and allows stopper to move up and down the obturator through rotation. During assembly, the stopper is threaded onto the back end of the obturator before the end cap is secured, allowing for precise adjustment of the obturator effective length (or penetration depth) by rotating the stopper. This subassembly is then inserted into the sheath such that the tip of the obturator protrudes. It should be noted that in an alternative embodiment, there can be an interlocking mechanism provided on the end of the obturator instead of the threaded portion; in this case, each of the stopper and the end cap can be properly configured to be attached to the end of the obturator by means of the interlocking mechanism. The sheath is an elastic tube capable of radially expanding outwards as an instrument is pushed through its inner diameter. The ribs can be stainless steel rods that are partially embedded within the elastic polymer body of the sheath. A portion of each rib member protrudes slightly from the elastic sheath, making contact with the instrument being inserted. This allows the ribs to serve as guide rails for any instrument inserted through the port sheath, allowing for a smooth instrument insertion. As a larger instrument is inserted through the inner diameter, it glides along the ribs, which in turn applies force to the sheath, causing the elastic polymer tube to expand radially outwards. The action of pushing a larger instrument through the sheath is what facilitates the radial expansion. This radial expansion mechanism allows for use of larger diameter instruments through the sheath while inducing minimal damage to the target anatomy. The ribs also act as support structures for the port sheath, preventing bending or kinking of the elastic port sheath when inserted into an anatomical structure such as a uterus. This allows the port sheath to maintain its shape while operating in its relevant anatomy. The entire assembly is inserted, for example, into the patient's abdomen at a 90° angle, and the obturator is removed, leaving the sheath behind.

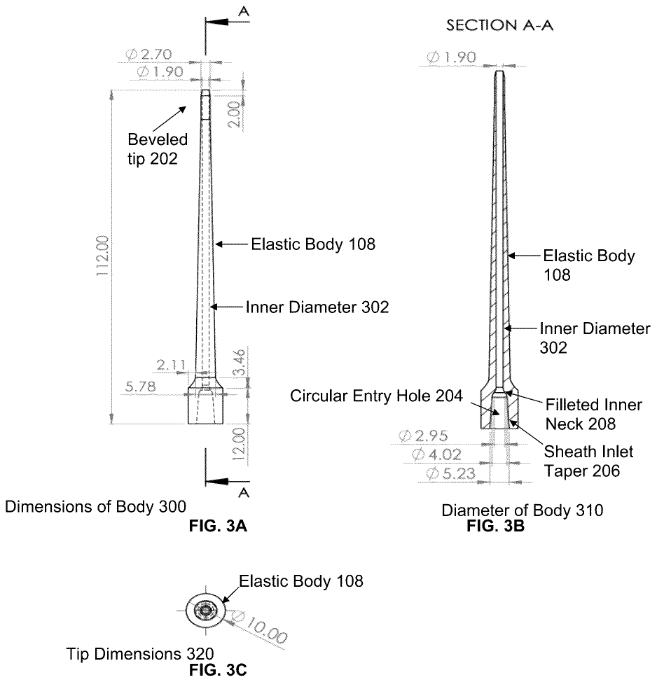

The sheath comprises a plurality of rigid ribs or supports encased in a flexible, elastic polymer tube. The ribs create a cylindrical morphology and reinforce the sheath throughout the surgery, preventing kinking during expansion as well as during patient movements, when layers of tissue can shift and create lateral shear forces. The ribs are encased end-to-end in polymer such that a fraction of the circumference of the rib is exposed inside the sheath. This allows instruments inserted through the sheath to make unobstructed contact with the ribs, acting as guide rails. The sheath morphology can be 2 mm in diameter, while it can expand to a 4 mm diameter with the insertion of larger instruments, as the elastic polymer stretches to accommodate the diameter of larger instruments. The sheath consists of a circular entry hole with a 5.2 mm initial inner diameter, a sheath inlet taper, a filleted inner neck, an elastic polymer body, and a beveled tip. The circular entry hole starts at a wide 5.2 mm and tapers down shallowly into a curved, filleted inner neck of the sheath. The shallow taper allows for an easy initial insertion of instruments into the entry hole while the rounded neck guides the instrument into the inner diameter of the sheath (see FIG. 3), allowing for a smoother insertion of instruments and an immediate radial expansion of the sheath. The port sheath has an outer diameter that starts at 5.78 mm and tapers down to 2 mm over the length of the elastic tube. The beveled tip of the sheath is designed to fit flush against the obturator, allowing for a smooth insertion into the target anatomy, preventing a “port step” as described above. The port sheath demonstrates a varying profile in the axial direction.

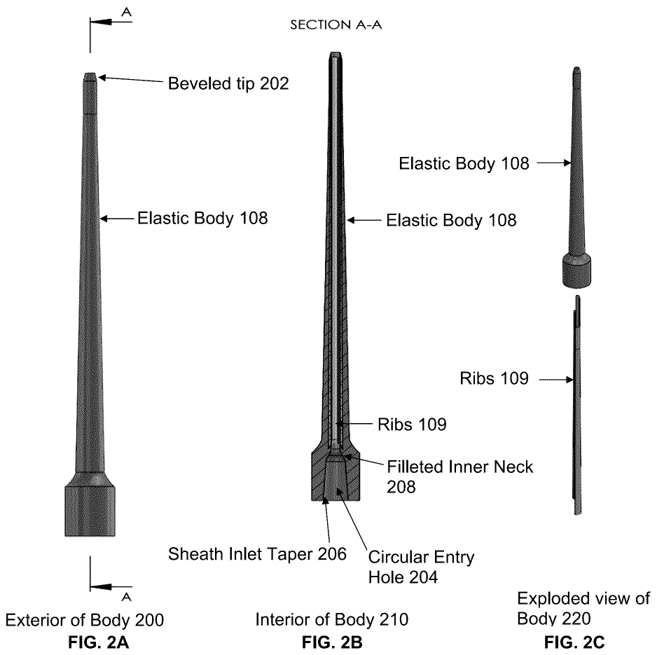

The sheath has a body made as an elastic polymer tube (like the sheath shown in FIG. 2C). The body is combined with the rigid ribs, and the entire sheath acts as a port system. The body of the sheath may have a beveled tip to create a flush profile when the obturator is inserted, as well as tapered walls to add strength. The sheath has a tapered neck that blends the instrument entry hole with the rest of the sheath body. This taper allows for the sheath to anchor onto its target anatomy, keeping it from protruding further into the target anatomy and causing unnecessary damage. When looking at a cross section of the sheath (see FIG. 3B), the sheath's outer diameter begins at about 5.2 mm, then tapers to and remains at a 2 mm inner diameter for the rest of the length of the sheath which is the minimum diameter of the sheath. The inner diameter of the sheath is about 2 mm (in an undeformed state, before expansion) for the entirety of the sheath. The sheath has an entry hole with a filleted inner neck for other surgical instruments such as the obturator to be inserted into. This filleted inner neck then transitions into the minimum sheath inner diameter which is maintained throughout the length of the sheath. This tapered inner minimum diameter stays constant throughout the length of the port until a larger instrument is inserted through the entry hole. Once a larger instrument is inserted through the entry hole, the instrument glides along the supporting ribs, applying force and causing the elastic polymer tube to conform to the diameter of the inserted instrument, expanding radially outward as a larger instrument is pushed through the vertical axis of the elastic polymer tube. The sheath demonstrates a varying profile shape along the length of the sheath in the axial direction. The ribs line the body of the sheath and maintain a uniformly straight shape throughout the length of the sheath, aside from a fillet on the end that helps the ribs evenly incorporate into the end of the elastic sheath.

Being partially embedded in the sheath, the ribs give it structure and support during the microsurgical procedures, preventing kinking of the sheath and aiding in radial expansion. Being partially embedded leaves one face of each rib exposed to the inner diameter of the sheath. As the ribs are more rigid, they act as rails for inserted instruments to slide along, and aid in expanding the sheath when a larger instrument is inserted. The overall rib profile is long and straight, with the end nearest the entry hole of the sheath being flared or chamfered, allowing inserted instruments to glide across, and the other end rounded off to reduce the risk of the ribs piercing the through the sheath. The ribs can be assembled in an evenly-spaced circular pattern in the sheath to create the cylindrical morphology. The rib is not uniformly straight in its shape; it features a fillet on the end to seamlessly incorporate into the entry hole of the elastic sheath. This fillet allows instruments to be inserted into the sheath without risk of striking and snagging the rib. The rib has an outer taper that follows the tapered profile of the sheath. In some cases, the ribs feature an arced profile, emphasizing the cylindrical morphology of the sheath profile. In some cases, the ribs features a triangular profile, which is easier to manufacture from standard stainless-steel rods.

In some cases, an obturator designed for use in controlled tissue penetration and sheath insertion may be made as a hardened stainless-steel rod, offering high durability and resistance to dulling of the cutting edge tip. The obturator may be 2 mm in diameter, optimized for precise insertion into small anatomy while minimizing trauma to surrounding tissues. The obturator has a finely sharpened, cutting-edge tip at one end and a threaded portion at another end. The cutting edge tip creates a linear wound profile upon insertion into the target anatomy. The cutting edge tip reduces tissue resistance upon entry into the target anatomy and ensures a clean, controlled insertion. This design reduces insertion force when entering target anatomy. The threaded portion may be provided on the last, e.g., 25 mm of the obturator and may configured as M2×0.4 threads. These threads allow the stopper and the end cap to be securely attached onto the body of the obturator. The obturator may undergo surface treatments such as passivation or titanium nitride coating to enhance corrosion resistance, reduce friction during insertion, and improve biocompatibility.

The tip may be specially designed to cut through the layers of tissue with minimal force and damage during insertion. The tip profile is made of four cut faces and four edges, creating 2 pairs of each. One pair of faces is offset at a shallower angle, creating the pair of longer edges and shorter edges. This tip profile of offset bevel edges acts like a dagger, as the shallower face can slice in a line, as opposed to piercing which occurs with identical faces. Each face extends 10 mm back from the tip, meaning the obturator must only protrude 10 mm from the sheath before they are radially flush. This makes insertion easier as there is a lower risk of striking the fetus with the tip during obturator insertion.

In some cases, the sheath may feature a tapered tip designed to blend seamlessly with the obturator. This tapered configuration may allow the sheath to transition smoothly onto the obturator rod, creating a continuous surface profile when the two components are assembled. The blended design may facilitate insertion of the port system by providing a smooth transition point between the obturator rod and the tip of the port sheath. This feature may help prevent snagging on surrounding anatomy or tissues during insertion, potentially reducing trauma to delicate structures. Additionally, the ribs incorporated within the sheath may be echogenic, enhancing visibility under ultrasound imaging. The echogenic properties of the ribs may improve the surgeon's ability to visualize and precisely position the access device during microsurgical procedures, particularly in applications where real-time imaging guidance is crucial.

The stopper may be made as a stainless-steel disk, e.g., with a M2×0.4 threaded through hole in the center that secures it to the obturator. It has a circumferential chamfer on both edges and concave engraved channels for easier gripping and handling with gloves. A surgeon can rotate the stopper, screwing it along the obturator threads, to adjust it upwards and downwards to change the effective length of the obturator, such that only the tip of the obturator protrudes from the sheath. The stopper acts to limit the obturator's forward movement through the sheath, eliminating the risk of striking tissue outside the target surgical site, such as striking the fetus with the obturator when performing fetal surgery. Limiting the movement of the obturator aids the surgeon as well, as the stopper can rest against the sheath's entry hole, thus the surgeon must only hold the sheath against obturator assembly. This effectively eliminates a degree of freedom, simplifying the sheath insertion process, as the surgeon need only press down on the end cap to insert the obturator and sheath together.

The end cap may be made of machined stainless-steel as a large convex knob, resembling a compressed sphere. Extending from the center of the end cap is a rounded extrusion which creates the end cap neck. In the center of the neck is a threaded hole which serves as the connection point to the obturator. The extended end cap neck creates a secure and reinforced attachment between the end cap and obturator, providing a smooth transition between the two, minimizing pressure points that could cause discomfort or instability during use. The end cap attaches to the end of the obturator, for example, with M2×0.4 threads. For an effective insertion, the surgeon places their palm on the end cap and pushes down; the subtle convexity of the end cap evenly distributes force across the surgeon's palm, allowing for a more precise application of force through the obturator, and a more precise sheath placement. The ergonomic end cap design helps prevent the surgeon from inserting the sheath at an angle, thus limiting the risk of hitting blood vessels, surrounding anatomy and organs, or inducing unnecessary stress on delicate tissues in the target surgical site, such as the amniotic membrane during instrument insertion in fetal surgery.

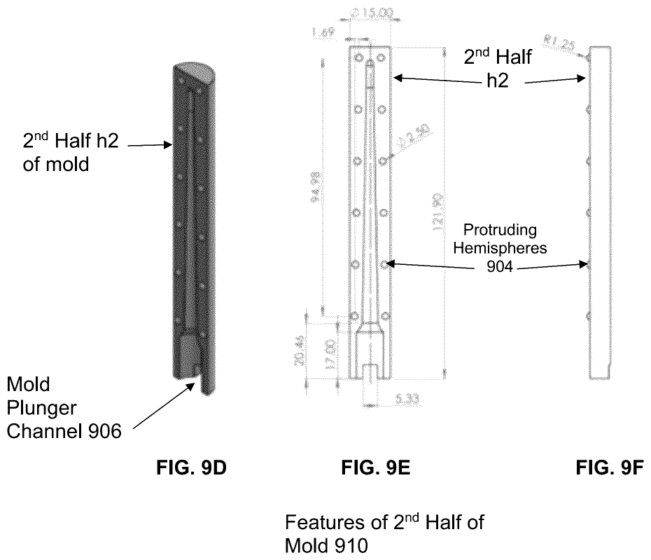

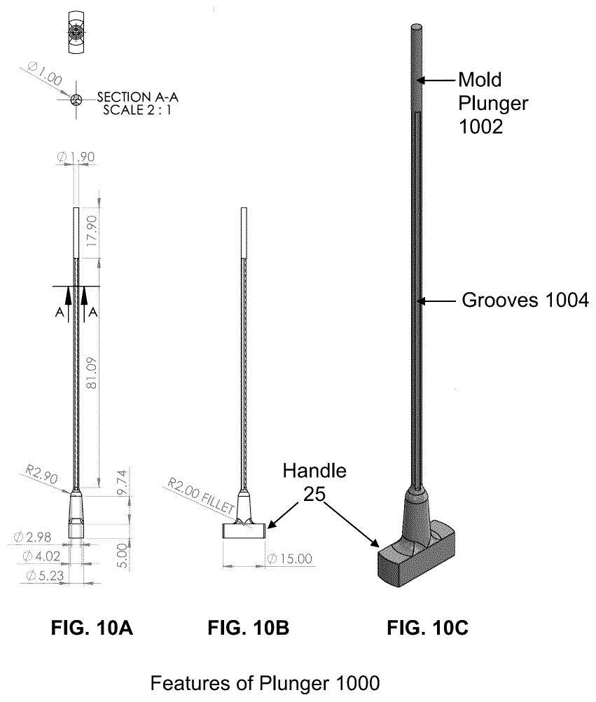

A mold for manufacturing the elastic body may include two halves. The mold can be used in the manufacturing of the elastic body of the sheath. Both halves fit together to create a negative space of the sheath. All the sizes in FIGS. 9A-9F are in millimeters. The second half features protruding hemispheres that mate into concave hemispheres in the first half, ensuring proper alignment of the two halves of the mold and creating a secure seal to prevent material leakage out of the mold. The first mold half contains a central cavity. This central cavity corresponds to half of the sheath shape. The second mold half contains a mirrored central cavity that corresponds to the other half of the sheath shape. When first and second halves are connected together by fitting the protruding hemispheres into the concave hemispheres, the full sheath geometry is achieved. There can be a mold plunger channel at the bottom of the mold, which fits a mold plunger (see FIGS. 10A-10C) to create the inner diameter of the sheath. This mold is designed for use with self-curing polymers; however, it could be adapted to work with injection molding or other manufacturing techniques. The mold halves may be coated with non-stick coating to facilitate the demolding process.

The mold plunger holds the ribs in place while the sheath is curing. There are grooves along the plunger body for the ribs to set in and be held in place during curing, and a long profile to create the inner cavity of the sheath. The top of the plunger has a small handle so that it can be easily grabbed and removed from the mold once the polymer has finished curing. The handle fits into the square channel of the mold. As the plunger is removed, it detaches from the ribs and leaves a negative mold of the inner diameter of the sheath.

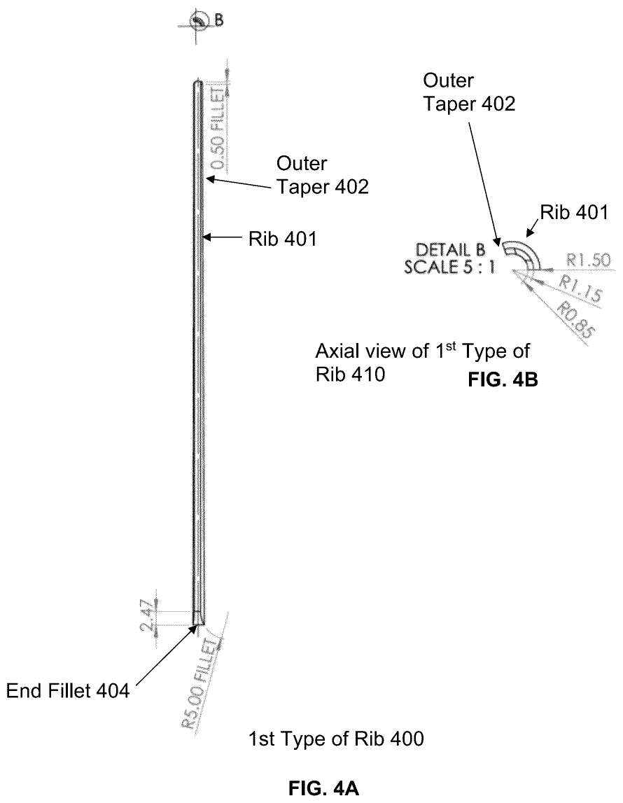

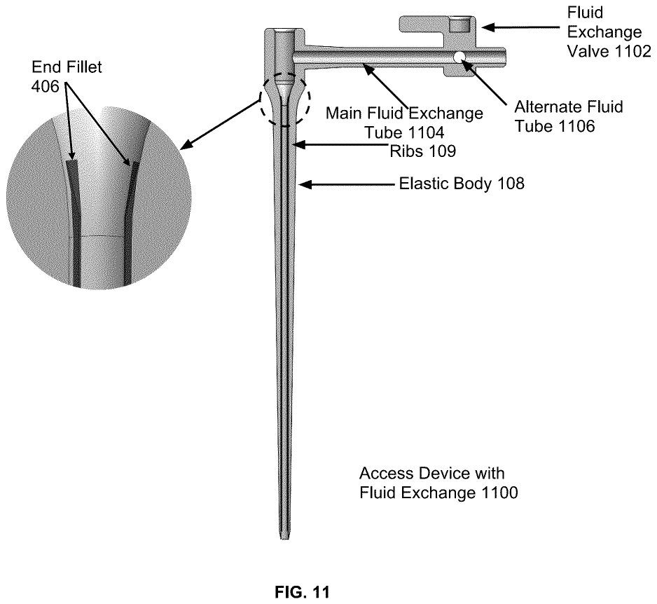

In some cases, the sheath features a fluid exchange valve. The valve is attached to the sheath to create a unibody construction, with a main fluid exchange tube leading to the three-way exchange valve and an alternate fluid tube. Rotating the lever on the valve allows the surgeon to open the fluid exchange valve to either, both, or neither of tubes. The lever blocks flow in the direction it is pointing, thus FIG. 11 is depicting a closed valve. This valve and tubing allows for fluid exchange in surgical procedures. For example, in fetal surgery the valve is used to remove amniotic fluid from the uterus at the start of the surgery, and to insufflate the uterus with humidified CO2 throughout the surgery. Humidified CO2 insufflation has been demonstrated to significantly benefit the outcome of the surgery, as it prevents the uterus from drying out during the surgery. FIG. 11 also gives a detailed view of the ribs embedded in the elastic body of the sheath, with a bevel or fillet towards the sheath entry hole. This fillet keeps instruments from striking the top of the ribs when inserted, instead sliding against the ribs and expanding the sheath. The ribs in FIG. 11 are the triangular embodiment of FIGS. 4C-4D.

In some views of the sheath, e.g., before expansion and after expansion, while inserted in a uterus, the uterus may be highly simplified, with one block representing all tissues from the skin to the amniotic membrane. In some cases, the sheath may feature a fluid exchange valve. Not pictured is an instrument that would be inserted in the sheath causing its expansion, as the sheath does not expand on its own. The sheath expands due to the force that an inserted larger instrument would exert onto the ribs. As a larger instrument is inserted into the sheath, it glides against the ribs, pushing outwards and causing the elastic polymer of the sheath to expand radially outwards. As the sheath expands, its inner diameter expands radially outward. The sheath inlet taper anchors the sheath in place within its target anatomy, preventing it from sliding too far into the target site.

In some cases, a sheath may feature a biocompatible inflatable balloon, which is inflated after insertion and aids to hold the amniotic membrane in place during the surgery. The biocompatible inflatable balloon has a spherical profile with rounded edges. When inflated and pressed against the amniotic membrane, the pressure makes the balloon take on a more elliptical profile. The balloon anchor fits flush to the exterior of the sheath when deflated and is inflated during surgery. Holding the amniotic membrane against the uterine wall keeps it from further separating during the surgery, as the movement of instruments into and out of the sheath can cause perturbations that strip the amniotic membrane off the uterine wall. The balloon can also be used for anchoring the sheath in some other surgical sites, including veins and/or arteries. The balloon is inflated by using the balloon inflation tube; the balloon inflation valve is opened by moving the valve lever to the balloon inflation valve position and then pushing a gas such as oxygen through the valve. As gas is pushed through the balloon inflation valve, the balloon inflates until it pushes up against the amniotic membrane, securing it to the uterine wall.

### 2. EMBODIMENTS

The present disclosure relates to an access device for surgical procedures. This device may provide improved access and control during various surgical interventions. In some cases, the access device may comprise multiple components working together to facilitate safe and effective entry into a surgical site.

The access device may include an obturator designed for controlled tissue penetration. A disc-shaped stopper may be incorporated to allow adjustment of the obturator's effective length. An end cap may be included to provide an ergonomic interface for the surgeon.

A key component of the access device may be a sheath with an elastic body. This sheath may be configured to expand radially, allowing the use of instruments of various sizes through a single access point. The elastic nature of the sheath body may help minimize trauma to surrounding tissues during instrument insertion and removal.

In some cases, the components of the access device may work in concert to provide a system for creating and maintaining surgical access while offering flexibility in instrument size and type. The device may be suitable for use in a variety of surgical procedures where controlled access to internal anatomy is desired.

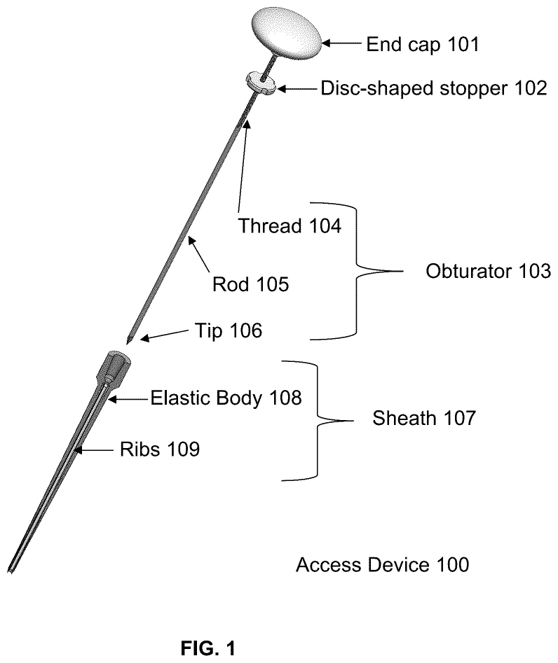

In some cases, an access device 100 for surgical procedures may include multiple components working together to facilitate controlled entry into a surgical site. FIG. 1 illustrates an isometric view of the access device 100, showing its main components and their arrangement.

The access device 100 may include an obturator 103. The obturator 103 may be a cylindrical rod that extends along a longitudinal axis. The obturator 103 may have a first end and a second end. At the first end, the obturator 103 may include a tip 106.

A disc stopper 102 may be attached to the second end of the obturator 103. The disc stopper 102 may be positioned at a first position along an axial length of the second end of the obturator 103.

An end cap 101 may also be attached to the second end of the obturator 103. The end cap 101 may be positioned at a second position along the axial length of the second end. This second position may be further away from the tip 106 than the first position of the disc stopper 102.

The obturator 103 may include a thread 104 along a portion of its length. The thread 104 may allow for adjustable positioning of the disc stopper 102. A rod 105 may extend distally from the thread 104 and terminate at the tip 106.

A sheath 107 may surround a portion of the rod 105. The sheath 107 may include an elastic body 108. The elastic body 108 may be configured as a radially expanding tube.

The elastic body 108 may include a set of ribs 109. The ribs 109 may extend axially along an inner surface of the elastic body 108. The ribs 109 may protrude radially inwardly from the inner surface. In some cases, the ribs 109 may be arranged circumferentially within the elastic body 108.

The ribs 109 may be configured to act as guide rails for the obturator 103. This configuration may allow the sheath 107 to pass the obturator 103 through the elastic body 108 along the ribs 109. The obturator 103 may be passed through until the disc stopper 102 rests against the elastic body 108.

The elastic body 108 may have a smooth outer surface. This smooth outer surface may allow the elastic body 108 to slide through tissue during insertion of the access device 100, minimizing frictional forces.

In some cases, the sheath 107 of the access device 100 may include several components that work together to facilitate insertion and expansion during use. FIG. 2A illustrates an orthogonal side view of a portion of the sheath 107, showing an exterior body 200 and a beveled tip 202.

The exterior body 200 may form the outer surface of the sheath 107. In some cases, the exterior body 200 may be made of an elastic, flexible, low-friction, biocompatible polymer. This material choice may allow the sheath 107 to expand radially while maintaining biocompatibility with surrounding tissues and minimizing damage to tissues during the surgery.

At the upper end of the sheath 107, the beveled tip 202 may be present. The beveled tip 202 may be designed to fit flush against the obturator 103 when inserted, creating a smooth transition between the obturator 103 and the sheath 107. This transition allows the access device 100 to be smoothly inserted into the target anatomy without snagging debris from surrounding tissues.

FIG. 2B provides a section view of the sheath 107, revealing internal components. A circular entry hole 204 may be present at the upper portion of the sheath 107. The circular entry hole 204 may transition into a sheath inlet taper 206. Below the sheath inlet taper 206, a filleted inner neck 208 may be present, leading to an interior body 210.

The interior body 210 may house the ribs 109, which may extend along the length of the sheath 107. The ribs 109 may protrude radially inward from the inner surface of the interior body 210, serving as guide rails for instruments passed through the sheath 107. The ribs 109 also serve to provide visibility of the sheath 107 on ultrasound imaging.

FIG. 2C presents an exploded view of a port body 220. The port body 220 may comprise the elastic body 108 and the ribs 109. This view illustrates how the ribs 109 may be integrated within the structure of the elastic body 108.

FIG. 3A and SECTION A-A provide dimensional details of the sheath 107. An inner diameter 302 may extend through the length of the elastic body 108. Body dimensions 300 may include a total length measurement, while a diameter of body 310 may represent the outer dimensional measurement of the elastic body 108.

In some cases, the sheath 107 may taper from about 5 millimeters at the top to about 2 millimeters at the bottom. This tapered design may contribute to the sheath's ability to expand from 2 millimeters to 4 millimeters in diameter when larger instruments are inserted.

FIG. 3C illustrates tip dimensions 320 of the sheath 107. The tip dimensions 320 may define the specific geometric parameters of the tapered section near the beveled tip 202, showing how the elastic body 108 transitions from its wider proximal portion to its narrower distal end.

The combination of these components—the exterior body 200, beveled tip 202, circular entry hole 204, sheath inlet taper 206, filleted inner neck 208, and interior body 210—may work together to create a sheath 107 capable of radial expansion while maintaining structural integrity during surgical procedures.

In some cases, the ribs 109 of the access device 100 may be configured in various ways to provide structural support and guide instruments through the sheath 107. FIGS. 4A-4D illustrate different rib configurations that may be incorporated into the elastic body 108 of the sheath 107.

FIG. 4A shows a first rib type 400. The first rib type 400 may include a rib 401 that extends longitudinally. The rib 401 may have an elongated straight profile along its length. An outer taper 402 may be present at one end of the rib 401. The outer taper 402 may provide a transition region that allows the rib 401 to integrate with the tapered profile of the sheath 107. An end fillet 404 may be provided at the opposite end of the rib 401, creating a rounded transition at that end. The rib type 400 material may be echogenic, making it visible on ultrasound imaging.

FIG. 4B presents a detail view of a portion of the access device 100, showing the rib 401 and outer taper 402. An axial rib 410 may be arranged longitudinally along the device. The rib 401 may have a triangular cross-sectional profile that protrudes inward from the inner surface of the sheath 107. This triangular profile may allow the tip of the rib 401 to extend into the inner diameter 302 of the sheath 107, acting as a guide rail for inserted instruments. The rib type 420 material may be echogenic, making it visible on ultrasound imaging.

FIG. 4C illustrates a section view of a second rib configuration. A longitudinal rib 425 may extend along the length of the sheath 107. The longitudinal rib 425 may include an end fillet 426 at its terminal portion. An outer taper 428 may be provided along the exterior edge of a second rib 420. The second rib 420 may have a generally triangular cross-sectional profile that protrudes inward from the inner diameter 302 of the sheath 107. The end fillet 426 and outer taper 428 allow the rib to easily integrate into the sheath 107 profile.

FIG. 4D shows an axial view of a second rib 430. The second rib 430 may feature an outer taper 428 that extends along its edge. This view displays how the outer taper 428 transitions from the main body of the second rib 430 to create a tapered profile.

In some cases, the ribs 109 may be distributed circumferentially about the inner surface of the elastic body 108. This arrangement may provide uniform support and guidance for instruments inserted through the sheath 107.

The flared or chamfered profile of the ribs 109, as shown by the outer taper 402 and outer taper 428, may be positioned where the obturator 103 enters the sheath 107. This configuration may allow for smoother insertion of the obturator 103 and other instruments into the sheath 107.

The rounded end of the ribs 109, as illustrated by the end fillet 404 and end fillet 426, may be located where the obturator 103 exits the sheath 107. This rounded profile may reduce the risk of the ribs 109 catching on or damaging instruments as they are withdrawn from the sheath 107, and reduce risk of them piercing through the sheath body 107 as instruments are inserted.

In some cases, the ribs 109 may have a cylindrical shape outlining the circumference of the sheath 107. This configuration may provide additional structural support to maintain the circular cross-section of the elastic body 108 during expansion and contraction.

The various rib configurations described may work in conjunction with the elastic body 108 to provide a balance of flexibility and structural integrity to the sheath 107. These configurations may allow the sheath 107 to expand radially while maintaining its overall shape and providing smooth guidance for inserted instruments. These various rib configurations described may be made of an echogenic material, allowing the sheath 107 to be visible on ultrasound imaging.

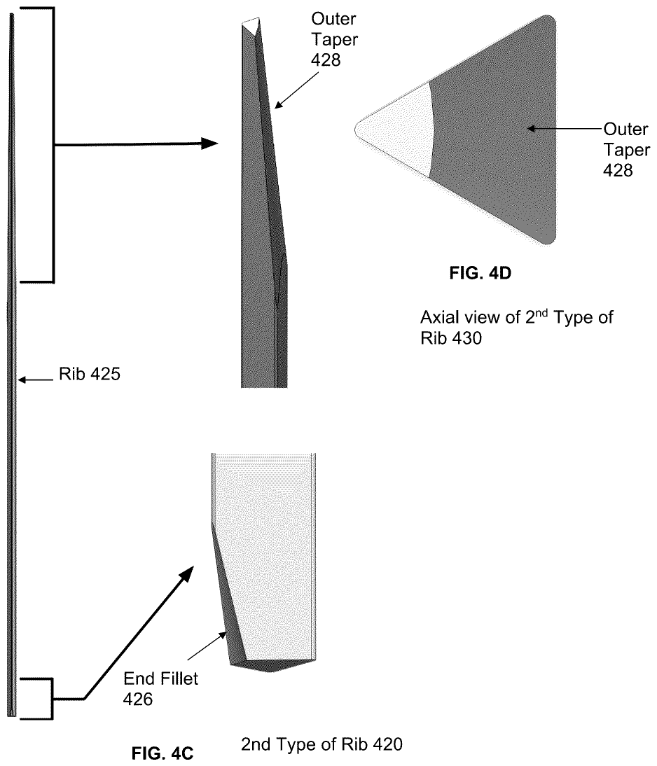

In some cases, the access device 100 may include an obturator 500 designed for controlled tissue penetration. FIG. 5 illustrates an orthogonal view of the obturator 500. The obturator 500 may include a rod 105 extending along its length and terminating in a tip 106 at its distal end. The rod 105 may be cylindrical in shape with a first end and a second end. The tip 106 may be located at the first end of the rod 105.

A locking mechanism 502 may be positioned at the proximal portion of the rod 105, near the second end. In some cases, the locking mechanism 502 may be a threaded portion. The threaded portion may have M2×0.4 threads. These threads may allow for adjustable attachment of the disc stopper 102 and the end cap 101 to the obturator 500.

In some cases, the second end of the rod 105 may have an interlocking mechanism instead of or in addition to the threaded portion. This interlocking mechanism may provide an alternative means of attaching the disc stopper 102 and the end cap 101 to the obturator 500.

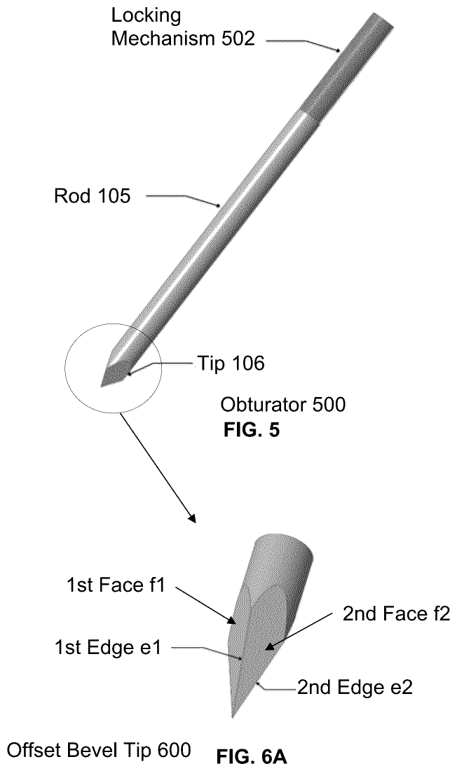

The tip 106 of the obturator 500 may be designed with an offset bevel configuration. FIG. 6A illustrates an orthogonal view of an offset bevel tip 600. The offset bevel tip 600 may comprise a first face f1 and a second face f2 that intersect to form a cutting edge. A first edge e1 may extend along one side of the first face f1, while a second edge e2 may extend along one side of the second face f2.

FIG. 6B provides a more detailed view of the offset bevel tip 610, showing additional geometric features. The offset bevel tip 610 may include four faces (f1, f2, f3, f4) and four edges (e1, e2, e3, e4) that form its geometric structure. The tip 106 may be formed by four cutting edges, comprising two long edges and two short edges. These long and short edges may alternate to create a tip profile that provides a linear wound during insertion.

The offset bevel tip 610 may feature three distinct lengths: a first length d1, a second length d2, and a third length d3. These lengths may be measured from the tip to different points along the edges. The varying lengths of these edges may contribute to the offset nature of the bevel design, enhancing the cutting performance of the tip 106.

The configuration of the offset bevel tip 610 with its multiple faces and edges of different lengths may allow for a more controlled and precise insertion of the obturator 500 into tissue. This design may help minimize tissue trauma during the insertion process while maintaining the effectiveness of the access device 100.

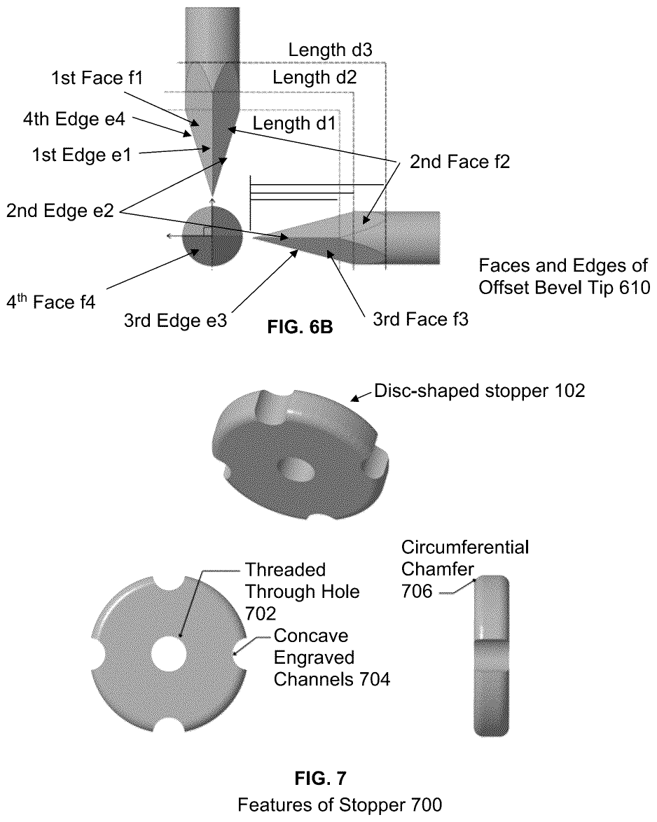

In some cases, the access device 100 may include a disc stopper 102 and an end cap 101 that attach to the obturator 103. FIG. 7 illustrates different views of a stopper assembly 700. The stopper assembly 700 may include the disc stopper 102 with a threaded hole 702 in its center. The threaded hole 702 may enable the disc stopper 102 to be threaded onto the thread 104 of the obturator 103.

The stopper assembly 700 may feature concave channels 704 arranged around its perimeter. These concave channels 704 may provide gripping surfaces for adjusting the position of the disc stopper 102. A circumferential chamfer 706 may be formed around the edge of the disc stopper 102, enhancing ease of handling.

In some cases, the threaded hole 702 may have M2×0.4 threads. This threading may allow for fine adjustment of the first position of the disc stopper 102 along the axial length of the obturator 103 by rotation.

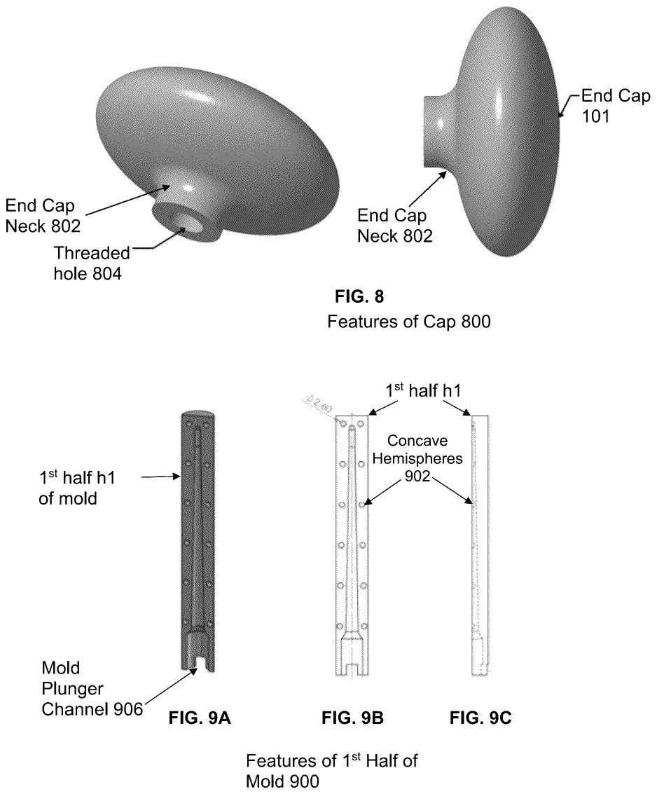

FIG. 8 illustrates orthogonal views of a cap 800, which may function as the end cap 101 for the access device 100. The cap 800 may include an end cap neck 802, which extends from the main body of the cap 800. A threaded hole 804 may be formed within the end cap neck 802, enabling attachment to the obturator 103.

In some cases, the cap 800 may be designed as a compressed spherical knob. The threaded hole 804 may be a dead threaded hole with M2×0.4 threads, configured to engage the thread 104 of the obturator 103.

The cap 800 may have a curved, dome-like shape designed to conform to a user's palm. This ergonomic design may allow for even distribution of force when the access device 100 is inserted into tissue.

In some cases, the disc stopper 102 and the end cap 101 may be configured to be locked in the first position and second position, respectively, by a locking mechanism 502 on the obturator 103. This locking mechanism 502 may be an alternative or addition to the threaded attachment method.

The disc stopper 102 may have a circumferential chamfer 706 on each edge and at least one concave channel 704 on its circumference. These features may enhance grip and ease of adjustment, particularly when wearing surgical gloves.

In some cases, the disc stopper 102 may incorporate an interlocking feature similar to a suitcase handle for adjustment. This feature may allow for quick and precise adjustment of the first position of the disc stopper 102 along the obturator 103.

The combination of the disc stopper 102 and the end cap 101 may allow for precise control of the obturator 103 insertion depth and provide an ergonomic interface for the user during surgical procedures.

In some cases, the sheath 107 of the access device 100 may be manufactured using a mold assembly 900 and a plunger assembly 1000. FIGS. 9A-9C illustrate different views of a first half of the mold assembly 900, while FIGS. 9D-9F show views of a second half h2 of a mold 910.

The mold assembly 900 may include an inner longitudinal channel shaped as the radially expanding tube of the sheath 107. This inner longitudinal channel may be represented by a plunger channel 906 that extends along the length of the mold assembly 900. The plunger channel 906 may be configured to receive and guide a mold plunger 1002 during the molding process.

The mold assembly 900 may be designed to be disassembled into two halves, with the plunger channel 906 divided along its longitudinal axis. This configuration may allow for easier removal of the molded sheath 107 after the manufacturing process.

In some cases, the first half of the mold assembly 900 may include concave hemispheres 902 arranged along its sides. These concave hemispheres 902 may be designed to align and mate with corresponding features on the second half h2 of the mold 910.

The second half h2 of the mold 910 may include protruding hemispheres 904 arranged on its surface. These protruding hemispheres 904 may be shaped and positioned to mate with the concave hemispheres 902 when the two halves of the mold assembly 900 are brought together. This mating of the hemispheres may help ensure proper alignment and secure closure of the mold during the manufacturing process.

FIGS. 10A-10C illustrate different views of the plunger assembly 1000 used in the manufacturing process. The plunger assembly 1000 may include a mold plunger 1002 that extends vertically from a base handle 25. The mold plunger 1002 may be shaped to be inserted into the plunger channel 906 of the mold assembly 900.

The mold plunger 1002 may include plunger grooves 1004 formed along its length. These plunger grooves 1004 may be configured to correspond to the shape of the ribs 109 in the sheath 107. The plunger grooves 1004 may be arranged longitudinally on the surface of the mold plunger 1002 and may be designed to hold the ribs 109 in place during the molding process.

In some cases, the manufacturing process may involve the following steps:

1. The mold assembly 900 may be prepared by aligning and joining the two halves, with the protruding hemispheres 904 mating with the concave hemispheres 902.

2. The mold plunger 1002 with ribs 109 attached to the plunger grooves 1004 may be inserted into the plunger channel 906 of the assembled mold.

3. A self-curing polymer may be injected into the space between the mold assembly 900 and the mold plunger 1002. This polymer may form the elastic body 108 of the sheath 107. The polymer will also secure the ribs 109 to the sheath 107.

4. After the self-curing polymer has cured, the mold plunger 1002 may be removed from the mold assembly 900.

5. The mold assembly 900 may then be disassembled into its two halves, releasing the newly formed sheath 107.

This manufacturing process may allow for the creation of a sheath 107 with integrated ribs 109, providing structural support and guidance for instruments inserted through the access device 100.

In some cases, the access device 1100 may include fluid exchange and expansion capabilities. FIG. 11 illustrates a section view of the access device 1100 with fluid exchange components.

The access device 1100 may include a fluid exchange valve 1102 connected to the sheath 107 at a region where the obturator 103 enters the sheath 107. The fluid exchange valve 1102 may be configured to provide fluid inlet and outlet through the sheath 107.

A main fluid exchange tube 1104 may extend through the length of the access device 1100. An alternate fluid tube 1106 may branch from the fluid exchange valve 1102. The fluid exchange valve 1102 may connect to both the main fluid exchange tube 1104 and the alternate fluid tube 1106, allowing for controlled fluid transfer through the access device 1100.

In some cases, the fluid exchange valve 1102 may allow exchange of both gas and fluid. The gas may be humidified CO2, while the fluid may be saline or amniotic fluid. This capability may enable surgeons to remove amniotic fluid from a uterus at the start of a surgery and insufflate the uterus with humidified CO2 throughout the procedure.

FIG. 11 also shows a detailed view of an end fillet 406 where the ribs 109 integrate with the elastic body 108. The end fillet 406 may allow instruments to be inserted into the sheath 107 without risk of striking or snagging the ribs 109.

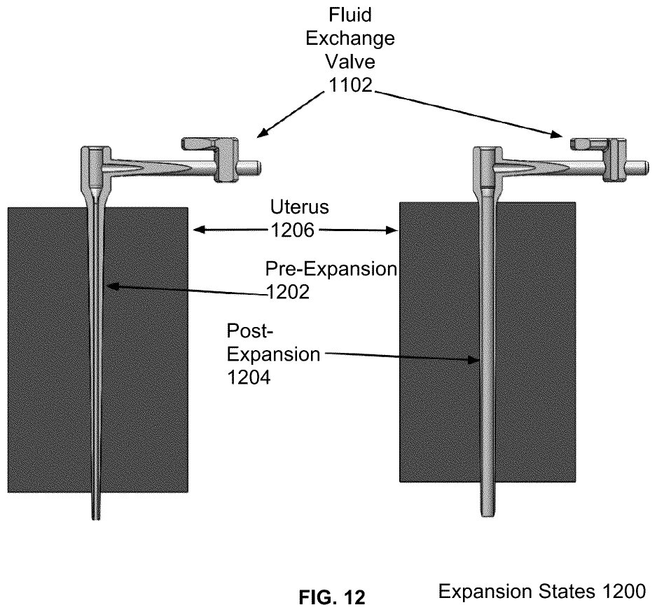

FIG. 12 illustrates expansion states 1200 of the access device 1100. The figure shows two states of the access device 1100 within a uterus 1206. On the left side, the access device 1100 is shown in a pre-expansion state 1202, where the sheath 107 has a smaller diameter. On the right side, the access device 1100 is depicted in a post-expansion state 1204, where the sheath 107 has expanded to accommodate a larger diameter instrument.

The fluid exchange valve 1102 may be positioned at the top of both configurations, allowing for fluid or gas exchange during use. The figure demonstrates how the access device 1100 may transition between different diameters while maintaining its position within the uterus 1206, with the fluid exchange valve 1102 remaining functional in both states.

In some cases, the sheath 107 may expand due to the force exerted by a larger instrument inserted through the sheath 107. As a larger instrument is inserted, the instrument may glide against the ribs 109, pushing outwards and causing the elastic body 108 of the sheath 107 to expand radially outwards. This expansion capability may allow surgeons to use instruments of various sizes through a single access point, minimizing tissue trauma during instrument insertion and removal.

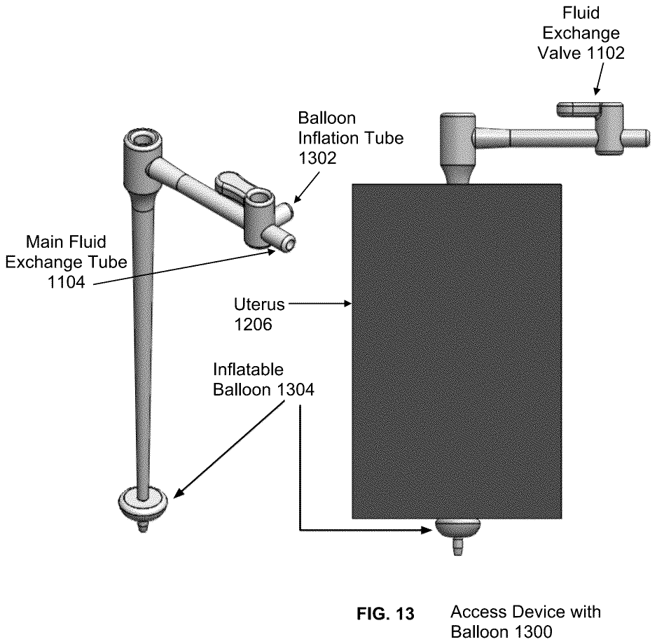

In some cases, the access device 1300 may include a balloon anchoring mechanism. FIG. 13 illustrates a side view of the access device 1300 with a balloon anchoring mechanism positioned within a uterus 1206.

The access device 1300 may include a balloon inflation tube 1302 that extends along the length of the device. The balloon inflation tube 1302 may connect to an inflatable balloon 1304 positioned near the distal end of the access device 1300.

The inflatable balloon 1304 may be configured to be inflated through the fluid exchange valve 1102. In some cases, the fluid exchange valve 1102 may allow for controlled delivery of fluid or air through either the main fluid exchange tube 1104 or the balloon inflation tube 1302. When fluid or air is directed through the balloon inflation tube 1302, the inflatable balloon 1304 may inflate or deflate as needed during use of the access device 1300.

The radially expanding tube of the sheath 107 may be self-anchored to surrounding anatomy through the inflatable balloon 1304. When inflated, the inflatable balloon 1304 may expand radially outward from the device body, contacting the inner wall of the uterus 1206. This contact may provide anchoring support for the access device 1300 within the surgical site.

In some cases, the radially expanding tube of the sheath 107 may have the biocompatible inflatable balloon 1304 at its tip. The inflatable balloon 1304 may have a spherical profile with rounded edges when inflated. When pressed against the surrounding anatomy, such as the amniotic membrane in a uterine procedure, the pressure may cause the inflatable balloon 1304 to take on a more elliptical profile.

The inflatable balloon 1304 may fit flush to the exterior of the sheath 107 when deflated. During a surgical procedure, the inflatable balloon 1304 may be inflated to anchor the access device 1300 in place. In uterine procedures, for example, the inflated balloon may hold the amniotic membrane against the uterine wall, preventing further separation during the surgery as instruments are moved in and out of the sheath 107.

The balloon anchoring mechanism may also be used for anchoring the access device 1300 in other surgical sites, including veins and/or arteries. The versatility of this anchoring mechanism may allow the access device 1300 to be used in a variety of surgical procedures where stable positioning within the target anatomy is desired.

In some cases, the access device may be used to perform surgical procedures by integrating its various components in a specific sequence of operations. This functional integration may allow for controlled access to a surgical site while minimizing tissue trauma.

The procedure may begin with the insertion of an obturator into a sheath. The obturator may be inserted such that a disc-shaped stopper attached to a second end of the obturator rests against an elastic body of the sheath. This configuration may allow for precise control of the obturator's insertion depth.

Access to a surgical site of interest may be provided by making a cut on a human body using the obturator inserted into the sheath. The combined obturator and sheath assembly may be advanced through the cut to reach the target surgical site.

Once the desired position is reached, the obturator may be removed from the sheath, while holding the sheath relative to the surgical site. This step may create a pathway for subsequent instrument insertion while maintaining access to the surgical site.

After obturator removal, a medical instrument of interest may be inserted into the surgical site through the sheath. The sheath's elastic body may accommodate instruments of various sizes, expanding radially as needed.

In some cases, the surgical procedure may be a fetal surgical procedure, and the surgical site of interest may comprise a uterus. The access device may be specifically designed to navigate the layers of tissue encountered during fetal surgery, including the uterine wall and amniotic membrane.

In other cases, the surgical site of interest may comprise at least one of a vein or an arteria. The access device may be adapted for use in vascular procedures, allowing for controlled entry into blood vessels while minimizing damage to vessel walls.

In other cases, the surgical site of interest may comprise at least one organ. The access device may be adapted for use in laparoscopic procedures, allowing for controlled entry into delicate organs or tissues while minimizing damage to surrounding anatomy.

The functional integration of the access device components may allow for a range of surgical applications, from fetal interventions to vascular procedures to laparoscopic procedures to neurosurgical interventions. The device may be especially useful when accessing small anatomy in pediatric patients. The device's design may enable surgeons to maintain stable access to the surgical site throughout the procedure, facilitating quick instrument exchange and minimizing the need for multiple entry points.

### 3. TERMINOLOGY

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the foregoing general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of +10% in a stated value.

The term “exemplary” is used in the sense of “example” rather than “ideal.” As used herein, the singular forms “a,” “an,” and “the” include plural reference unless the context dictates otherwise.

### 4. EXAMPLES

Exemplary embodiments of the systems and methods disclosed herein are described in the numbered paragraphs below.

A1. An access device for surgical procedures, the access device comprising: an obturator, wherein the obturator is a cylindrical rod that has a first end and a second end, and the first end has a tip; a disc-shaped stopper, wherein the disc-shaped stopper is configured to be attached to the second end of the obturator at a first position along an axial length of the second end; an end cap, wherein the end cap is configured to be attached to the second end of the obturator at a second position along the axial length of the second end, and the second position is further away from the tip of the first end than the first position; and a sheath having an elastic body, wherein the elastic body includes: a radially expanding tube, and a set of echogenic reinforcing ribs extending axially along an inner surface of the radially expanding tube and protruding radially inwardly from the inner surface of the radially expanding tube, wherein the set of reinforcing ribs are configured to act as guide rails for the obturator, maintain the structural integrity of the port during shear/kinking forces, and be visible on ultrasound, and the radially expanding tube has a smooth outer surface to cause the radially expanding tube to slide through a tissue of interest, and wherein the sheath is configured to pass the obturator through the radially expanding tube along the set of reinforcing ribs until the disc-shaped stopper attached to the second end of the obturator rests against the radially expanding tube.

A2. The access device of A1, wherein the second end of the cylindrical rod has a threaded portion, and wherein the disc-shaped stopper and the end cap are configured to be threaded onto the threaded portion.

A3. The access device of A2, the disc-shaped stopper has a central threaded hole passing therethrough configured to engage the threaded portion, to thereby enable adjustment of the first position by rotation of the disc-shaped stopper.

A4. The access device of A3, wherein the end cap is a compressed spherical knob having a dead threaded hole, and the dead threaded hole is configured to engage the threaded portion.

A5. The access device of any of A1-A4, wherein the second end has an interlocking mechanism, and the disc-shaped stopper and the end cap are configured to be locked in the first position and second position, respectively, by the interlocking mechanism.

A6. The access device of any of A1-A5, wherein the disc-shaped stopper has a circumferential chamfer on each edge and at least one concave channel on a circumference of the disc-shaped stopper.

A7. The access device of any of A1-A6, wherein the tip of the first end is formed by four cutting edges comprising two long edges and two short edges, and the long edges alternate with the short edges so as to create a tip profile that provides a linear wound during insertion.

A8. The access device of any of A1-A7, wherein each reinforcing rib of the set of reinforcing ribs has an elongated straight profile that is flared or chamfered at a point where the obturator enters the sheath and rounded at a point where the obturator exits the sheath.

A9. The access device of any of A1-A8, wherein the set of reinforcing ribs are distributed circumferentially about the inner surface.

A10. The access device of any of A1-A9, wherein the radially expanding tube is made of an elastic, flexible, low-friction, biocompatible polymer.

A11. The access device of any of A1-A10, wherein the radially expanding tube has a tapered wall and a beveled tip, wherein the beveled tip fits flush against the obturator when the obturator is inserted into the radially expanding tube.

A12. The access device of any of A1-A11, further comprising a fluid exchange valve connected to the sheath at a region where the obturator enters the sheath, and the fluid exchange valve is configured to provide fluid inlet and outlet through the sheath.

A13. The access device of A12, wherein the radially expanding tube is configured to be self-anchored to a surrounding anatomy through a biocompatible inflatable balloon provided at a tip of the radially expanding tube, the biocompatible inflatable balloon being configured to be inflated through the fluid exchange valve.

B1. An obturator comprising: a cylindrical rod that has a first end and a second end, wherein the first end has a tip; a disc-shaped stopper, wherein the disc-shaped stopper is configured to be attached to the second end of the obturator at a first position along an axial length of the second end; and an end cap, wherein the end cap is configured to be attached to the second end of the obturator at a second position along the axial length of the second end, and the second position is further away from the tip of the first end than the first position.

C1. An access device for surgical procedures, the access device comprising: a sheath having an elastic body, wherein the elastic body includes: a radially expanding tube, and a set of echogenic reinforcing ribs extending axially along an inner surface of the radially expanding tube and protruding radially inwardly from the inner surface of the radially expanding tube, wherein the set of reinforcing ribs are configured to act as guide rails for an obturator and/or surgical devices and are visible on ultrasound imaging, and the radially expanding tube has a smooth outer surface to cause the radially expanding tube to slide through a tissue of interest, and wherein the sheath is configured to pass the obturator and/or the surgical devices through the radially expanding tube along the set of reinforcing ribs.

D1. A method for manufacturing a sheath of an access device, the method comprising: preparing a mold for a sheath, wherein the mold has an inner longitudinal channel shaped as a radially expanding tube of the sheath, the inner longitudinal channel has a longitudinal axis, and the mold is configured to be disassembled into two halves such that the inner longitudinal channel is divided in half along the longitudinal axis of the channel; preparing a mold plunger, wherein the mold plunger is shaped to be inserted into the inner longitudinal channel of the mold, and the mold plunger includes longitudinal grooves each configured to correspond to a shape of one of the set of reinforcing ribs; inserting the mold plunger into the mold; injecting a self-curing polymer into a space between the mold and the mold plunger; and after the self-curing polymer is cured, removing the mold plunger and disassembling the mold into the two halves, thereby releasing the sheath.

D2. The method of D1, wherein a first one of the two halves comprises protruding hemispheres, a second one of the two halves comprises concave hemispheres, and the protruding hemispheres and the concave hemispheres are shaped and positioned to mate with each other when the two halves are assembled together.

E1. A method for performing a surgical procedure by using an access device, the method comprising: inserting an obturator into a sheath such that a disc-shaped stopper attached to a second end of the obturator rests against an elastic body of the sheath; providing access to a surgical site of interest by making a cut on a human body using the obturator inserted into the sheath; removing the obturator from the sheath, while holding the sheath relative to the surgical site; and inserting a medical instrument of interest into the surgical site through the sheath.

E2. The method of E1, wherein the surgical procedure is a fetal surgical procedure, and the surgical site of interest comprises a uterus.

E3. The method of E1, wherein the surgical site of interest comprises at least one of a vein, an arteria, or an abdomen.

Other aspects of the disclosure will be apparent to those skilled in the art from consideration of the specification and practice of the invention disclosed herein. It is intended that the specification and examples be considered as exemplary only, with a true scope and spirit of the invention being indicated by the following claims.

1. An access device for surgical procedures, the access device comprising:
an obturator, wherein the obturator is a cylindrical rod that has a first end and a second end, and the first end has a tip;
a disc-shaped stopper, wherein the disc-shaped stopper is configured to be attached to the second end of the obturator at a first position along an axial length of the second end;
an end cap, wherein the end cap is configured to be attached to the second end of the obturator at a second position along the axial length of the second end, and the second position is further away from the tip of the first end than the first position; and
a sheath having an elastic body, wherein the elastic body includes:
a radially expanding tube, and
a set of echogenic reinforcing ribs extending axially along an inner surface of the radially expanding tube and protruding radially inwardly from the inner surface of the radially expanding tube,
wherein the set of reinforcing ribs are configured to act as guide rails for the obturator, maintain the structural integrity of the port during shear/kinking forces, and be visible on ultrasound, and the radially expanding tube has a smooth outer surface to cause the radially expanding tube to slide through a tissue of interest, and
wherein the sheath is configured to pass the obturator through the radially expanding tube along the set of reinforcing ribs until the disc-shaped stopper attached to the second end of the obturator rests against the radially expanding tube.

2. The access device of claim 1, wherein the second end of the cylindrical rod has a threaded portion, and wherein the disc-shaped stopper and the end cap are configured to be threaded onto the threaded portion.

3. The access device of claim 2, the disc-shaped stopper has a central threaded hole passing therethrough configured to engage the threaded portion, to thereby enable adjustment of the first position by rotation of the disc-shaped stopper.

4. The access device of claim 3, wherein the end cap is a compressed spherical knob having a dead threaded hole, and the dead threaded hole is configured to engage the threaded portion.

5. The access device of claim 1, wherein the second end has an interlocking mechanism, and the disc-shaped stopper and the end cap are configured to be locked in the first position and second position, respectively, by the interlocking mechanism.

6. The access device of claim 1, wherein the disc-shaped stopper has a circumferential chamfer on each edge and at least one concave channel on a circumference of the disc-shaped stopper.

7. The access device of claim 1, wherein the tip of the first end is formed by four cutting edges comprising two long edges and two short edges, and the long edges alternate with the short edges so as to create a tip profile that provides a linear wound during insertion.

8. The access device of claim 1, wherein each reinforcing rib of the set of reinforcing ribs has an elongated straight profile that is flared or chamfered at a point, on a first side of the reinforcing rib, where the obturator enters the sheath and rounded at a point, on a second side of the reinforcing rib opposite the first side, where the obturator exits the sheath.

9. The access device of claim 1, wherein the set of reinforcing ribs are distributed circumferentially about the inner surface.

10. The access device of claim 1, wherein the radially expanding tube is made of an elastic, flexible, low-friction, biocompatible polymer.

11. The access device of claim 1, wherein the radially expanding tube has a tapered wall and a beveled tip, wherein the beveled tip fits flush against the obturator when the obturator is inserted into the radially expanding tube.

12. The access device of claim 1, further comprising a fluid exchange valve connected to the sheath at a region where the obturator enters the sheath, and the fluid exchange valve is configured to provide fluid inlet and outlet through the sheath.

13. The access device of claim 12, wherein the radially expanding tube is configured to be self-anchored to a surrounding anatomy through a biocompatible inflatable balloon provided at a tip of the radially expanding tube, the biocompatible inflatable balloon being configured to be inflated through the fluid exchange valve.

14. An obturator comprising:
a cylindrical rod that has a first end and a second end, wherein the first end has a tip;
a disc-shaped stopper, wherein the disc-shaped stopper is configured to be attached to the second end of the obturator at a first position along an axial length of the second end; and
an end cap, wherein the end cap is configured to be attached to the second end of the obturator at a second position along the axial length of the second end, and the second position is further away from the tip of the first end than the first position.

15. An access device for surgical procedures, the access device comprising:
a sheath having an elastic body, wherein the elastic body includes:
a radially expanding tube, and
a set of echogenic reinforcing ribs extending axially along an inner surface of the radially expanding tube and protruding radially inwardly from the inner surface of the radially expanding tube,
wherein the set of reinforcing ribs are configured to act as guide rails for an obturator and/or surgical devices and are visible on ultrasound imaging, and the radially expanding tube has a smooth outer surface to cause the radially expanding tube to slide through a tissue of interest,
wherein the sheath is configured to pass the obturator and/or the surgical devices through the radially expanding tube along the set of reinforcing ribs,
wherein each reinforcing rib of the set of reinforcing ribs has an elongated straight profile that is flared or chamfered at a point, on a first side of the reinforcing rib, where the obturator enters the sheath and rounded at a point, on a second side of the reinforcing rib opposite the first side, where the obturator exits the sheath, and
wherein the set of reinforcing ribs are partially embedded within the elastic body such that a portion of each rib protrudes radially inwardly from the inner surface.

16. (canceled)

17. (canceled)

18. A method for performing a surgical procedure by using an access device, the method comprising:
inserting an obturator into a sheath such that a disc-shaped stopper attached to a second end of the obturator rests against an elastic body of the sheath;
providing access to a surgical site of interest by making a cut on a human body using the obturator inserted into the sheath;
removing the obturator from the sheath, while holding the sheath relative to the surgical site; and
inserting a medical instrument of interest into the surgical site through the sheath.

19. The method of claim 18, wherein the surgical procedure is a fetal surgical procedure, and the surgical site of interest comprises a uterus.

20. The method of claim 18, wherein the surgical site of interest comprises at least one of a vein, an arteria, or an abdomen.
